(19) United States
(12) Patent Application Publication
(10) Pub. No.: US 2016/0376086 A1
(43) Pub. Date: Dec. 29, 2016

(54) Glove Storage and Dispensing System

(71) Applicant: Hankscraft, Inc., Reedsburg, WI (US)

(72) Inventors: Kelly G. Damaschke, Loganville, WI (US); Joseph J. Hennessy, Reedsburg, WI (US)

(21) Appl. No.: 14/747,715

(22) Filed: Jun. 23, 2015

(51) Int. Cl. B65D 83/08; A41D 19/00; A61B 42/40

(52) U.S. Cl. CPC B65D 83/08; A61B 42/40; A41D 19/0055

(57) ABSTRACT

A system stores and dispenses gloves. The system includes a glove support configured to hold and support a plurality of gloves, and a glove dispensing housing configured to receive the glove support. The glove support includes a mating tab that forms an interference fit with detents in the glove dispensing housing. A user inserts their hand into a top-most glove in a stack of gloves in the glove support, and tears the glove away from the glove support. Securing tabs and pins aid the user's tearing action by providing resistance against the motion.

### INTRODUCTION

Direct skin contact between a person's fingers or hands and a surface is undesirable in many instances. For example, in public restrooms, the door handles can host bacteria deleterious to a person's health. In surgical theater, medical personnel wear protective gear including gloves. Personnel preparing food for sale or public consumption often wear protective gloves.

### SUMMARY

In general terms, this disclosure is directed to a glove dispensing system that includes a glove dispensing housing, a glove support, and one or more gloves.

In one aspect, a glove support includes a body with a planar surface including a mating tab, where the mating tab is configured to form an interference fit with a detent on a housing and where the body is configured to support a plurality of gloves. The glove support also includes at least one holding tab that is integral with the body, where the tab has a planar surface that is substantially parallel to the planar surface of the body.

In another aspect, a glove dispensing system includes a body with a planar surface including a mating tab, where the mating tab includes at least two outer edge surfaces, where the at least two outer edge surfaces are substantially normal to the planar surface of the body, and where the at least two outer edge surfaces are substantially parallel to each other. The glove dispensing system also includes at least one holding tab that is integral with the body and has a planar surface that is substantially parallel to the planar surface of the body, where the body is configured to support a plurality of gloves, and where at least one glove is supported by the body.

In yet another aspect, a glove tray includes a body with an insert tab, a first resistance tab, a second resistance tab, and a third resistance tab. The insert tab is integral to the body and configured to form an interference fit with a securing component. The body is substantially planar. Each of the first resistance tab, the second resistance tab, and the third resistance tab are substantially planar, supported by the body, and parallel to the planar surface of the body.

### BRIEF DESCRIPTION OF THE DRAWINGS

The following figures, which form a part of this application, are illustrative of described technology and are not meant to limit the scope of the claims in any manner, which scope shall be based on the claims appended hereto.

FIG. 1 is a schematic block diagram of an example glove dispensing system.

FIG. 2 is a front perspective view of an example glove dispensing housing.

FIG. 3 is a rear perspective view of the example glove dispensing housing of FIG. 2.

FIG. 4 is an illustration of an example housing saddle, glove support and glove.

FIG. 5 is a perspective view of the example glove support and glove of FIG. 4.

FIG. 6 is a perspective view of an example detent of the example housing saddle shown in FIG. 4.

FIG. 7 is a front view of the detent of the example glove dispensing housing of FIG. 2.

FIG. 8 is a front view of an alternate embodiment of a detent.

FIG. 9 is a perspective view of a second alternate embodiment of a detent.

FIG. 10 is a front elevational view of a first alternate embodiment of a glove support.

FIG. 11 is a front elevational view of a second additional alternate embodiment of a glove support.

FIG. 12 is a front elevational view of the second additional alternate embodiment of a glove support for a left-handed glove.

FIG. 13 is a front elevational view of an example glove.

FIG. 14 is a front elevational view of an example left-handed glove.

FIG. 15 is a detail view of a pre-mated example glove support and housing saddle.

FIG. 16 is a detail view of a mated example glove support and housing saddle.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

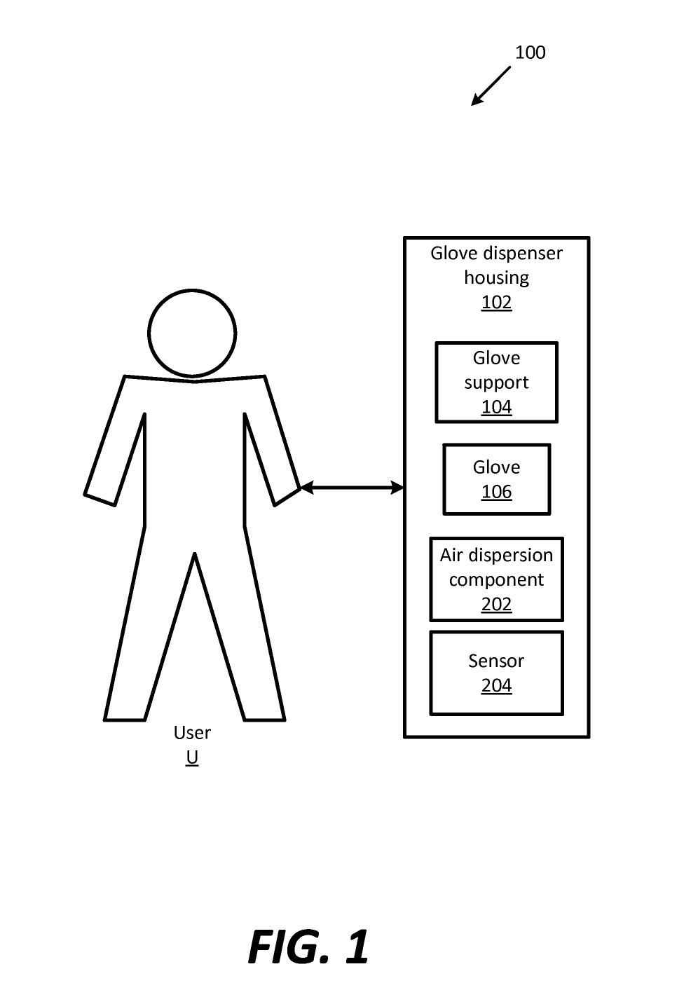

FIG. 1 is a schematic block diagram of an example glove dispensing system 100. The system 100 includes a glove dispensing housing 102, which includes an interface with a glove support 104 that supports a plurality of gloves 106, an air dispersion component 202, and a sensor 204. Also depicted is a user U. Other examples can include more or fewer components.

The user U can be a person who is required or who wishes to don a glove. In some instances, it may be beneficial or necessary to don the glove without touching the outside of the glove. For example, the user U is a person in a public restroom and the glove dispensing housing 102 is positioned such that the user U can don a glove and open the door without actually touching the door handle with their hand. Also, the user U can be a person working in foodservice or in a medical environment, such as an exam room or an operating theater. Applications in other circumstances and industries are possible.

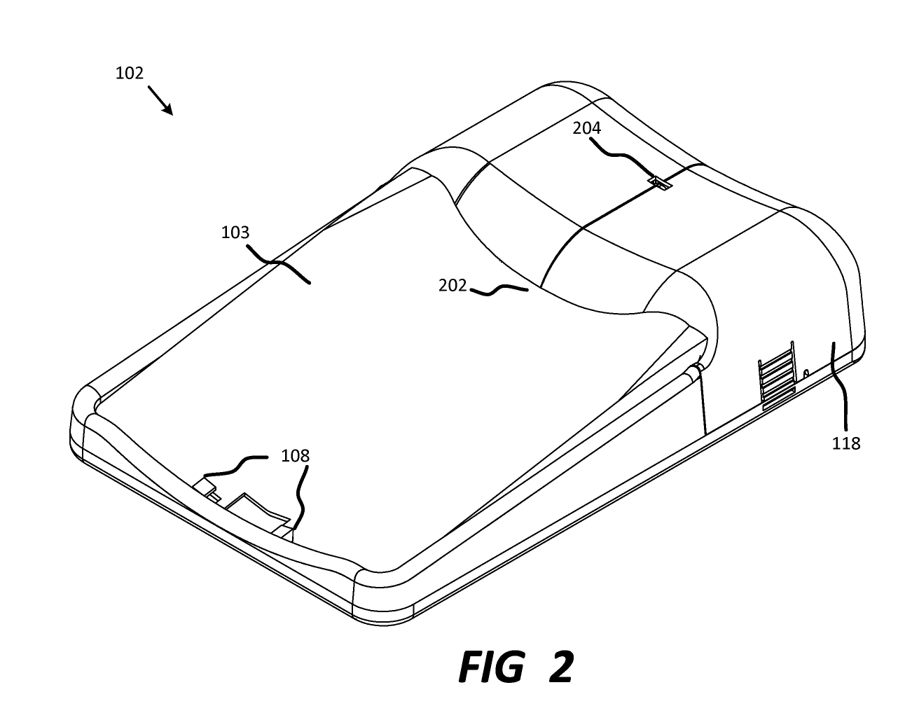

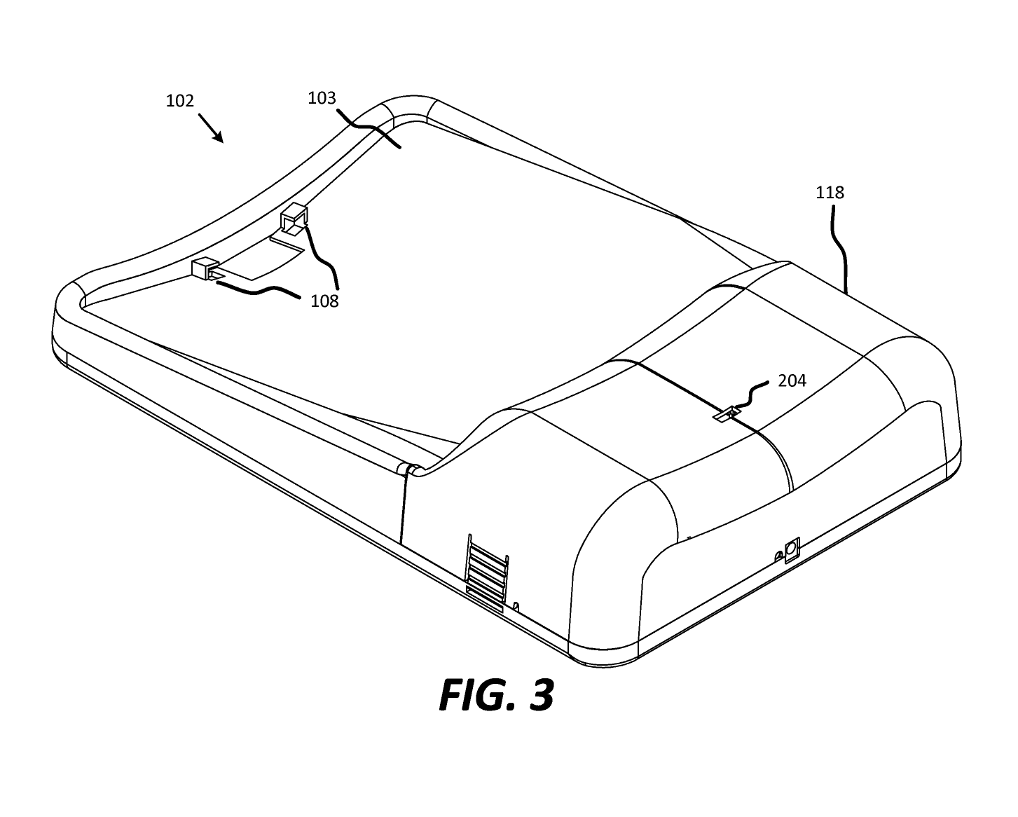

The glove dispensing housing 102 includes a receiving portion, which can be a cavity or indentation that is sized to receive a glove support 104. An example glove dispensing housing 102 is shown and described below with reference to FIGS. 2 and 3. The glove dispensing housing 102 includes a housing saddle 103 that includes features that interface with features on the glove support 104. Examples of these interfaces are shown and described below.

The glove support 104 is a substantially planar surface that provides a support for the plurality of gloves 106. The glove support 104 is constructed of a rigid or semi-rigid material. Example materials include plastics, cardboard, paper board, particle board, and metals. Other materials are possible.

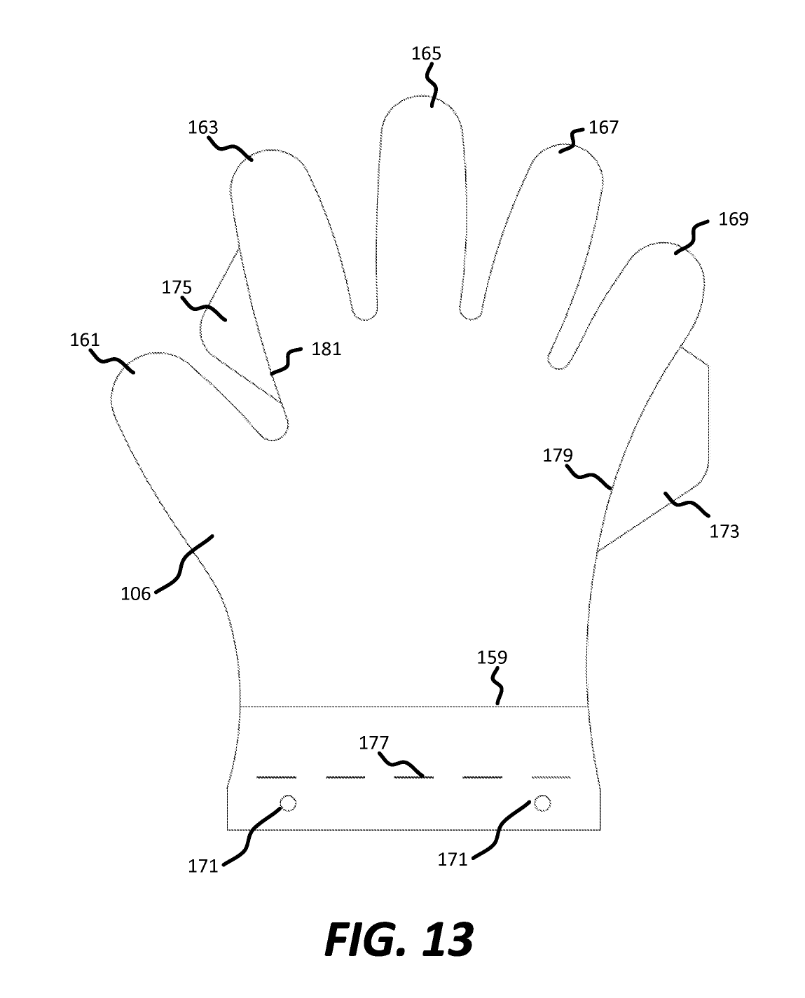

Gloves 106 may be a group or stack of disposable gloves. However, non-disposable glove uses are possible. Gloves 106 can be made from polyethylene, silicone, nitrile, vinyl, latex, and neoprene. In some embodiments, gloves 106 include one or more tear or perforation lines, shown and described below with reference to FIG. 13. In those embodiments, the user U inserts their hand into the glove and then applies a force to cause the glove to tear away along the perforation lines.

In embodiments, the user U is assisted by an air dispersion component 202 supported by the glove dispensing housing 102. The air dispersion component 202 can be activated without touch, such as by waving a hand and having the motion detected by a motion sensor 204. When the air dispersion component is activated, pressurized air is emitted from the air dispersion component 202 and directed into the top-most glove, causing the top-most glove to inflate. The user U then inserts their hand into the partially to fully-inflated glove. Then the user U pulls their gloved hand away from the glove dispensing housing 102. In this way, the user U is able to don the glove without touching the outer surface of the glove.

FIGS. 2 and 3 illustrate an example glove dispensing housing 102. The example glove dispensing housing 102 includes housing saddle 103 with detents 108, cover 118, air dispersion component 202, and motion sensor 204. Other embodiments can include more or fewer components and have a different configuration. Additional examples of glove dispensing housing 102 are described in U.S. Pat. Nos. 8,146,776, 8,651,323, and 8,657,151, the entirety of which are hereby incorporated by reference.

As shown, housing saddle 103 is a curved surface with a substantially v-shaped cross-section. In other embodiments, housing saddle 103 is substantially flat. Detents 108 are described in more detail below.

The cover 118 is connected to the glove dispensing housing 102 by a hinge and can be opened and closed. Cover 118 hides and protects components of glove dispensing housing 102, including a power source, such as batteries or a power conversion unit, air dispersion component 202, receiving cavities (shown and described below), and control unit. There exists an opening between the cover 118 and the housing saddle 103 through which air can pass from the air dispersion component 202 into the glove 106.

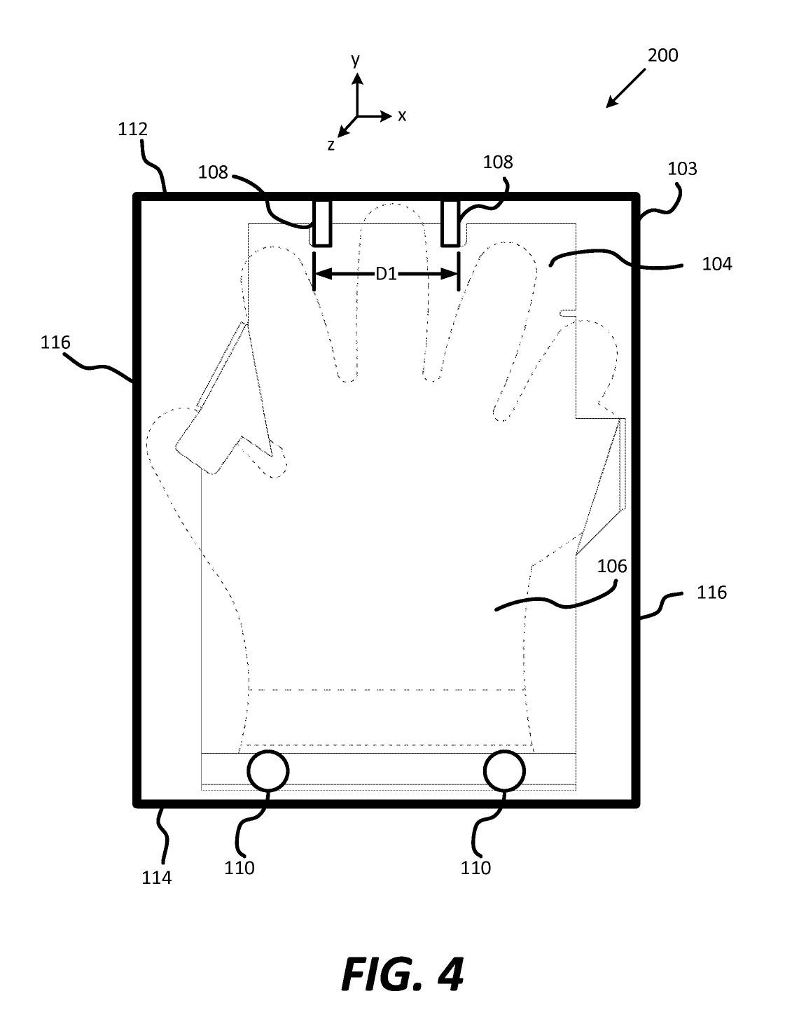

FIG. 4 illustrates an example environment 200 that includes a housing saddle 103 with detents 108, glove support 104, and glove 106. Also shown are receiving cavities 110 for securing pins, distance D1 between the top detents, top edge surface 112 of the housing saddle 103, bottom edge surface 114 of the housing saddle 103, and side edges 116 of the housing saddle. Other embodiments can include more or fewer components. The glove support 104 and glove 106 are described below in more detail.

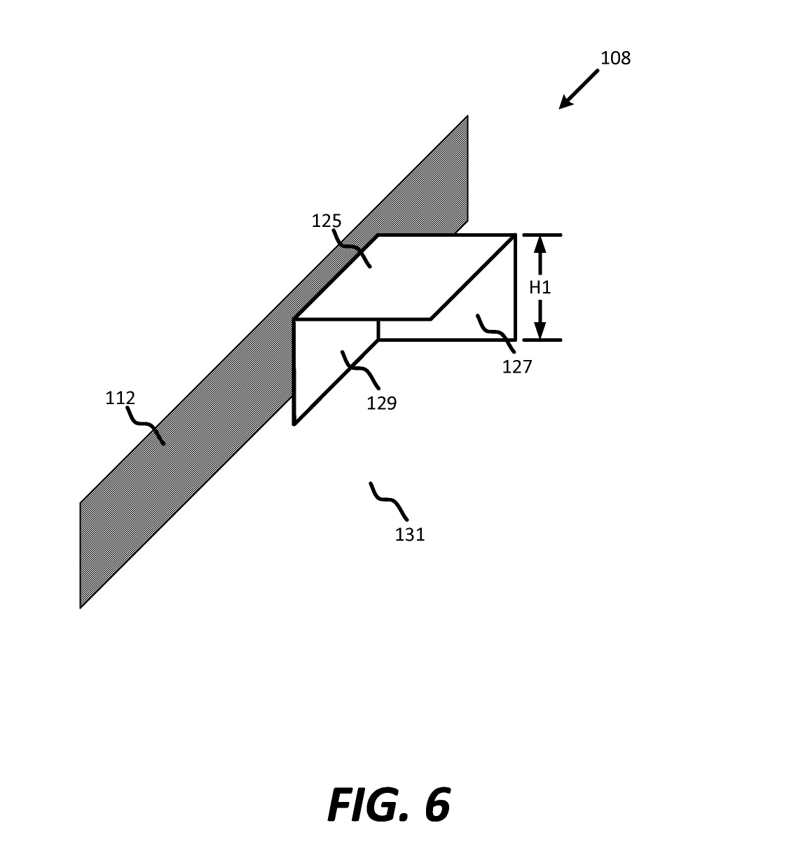

Housing saddle 103 is integral with the glove dispensing housing 102 and includes one or more mating features. The one or more mating features can include two detents 108. In the example, detents 108 are positioned adjacent to the top edge surface 112 of the housing saddle 103. In other embodiments, detents 108 are positioned adjacent to the side surfaces 116 of the housing saddle 103. In embodiments, detents 108 have rounded edges and/or curved surfaces. Example detents 108 are shown and described in more detail below with reference to FIGS. 6-9.

Detents 108 include a substantially planar surface, in the y-z plane, that is orthogonal to the plane of the housing saddle 103, in the x-y plane. The surfaces in the y-z plane of the detents 108 inhibit lateral movement of the glove support 104 in the x-y plane. Detents 108 also include a substantially planar surface in the x-y plane that is substantially parallel to the x-y plane of the housing saddle 103. The surfaces in the x-y plane of the detents 108 limit vertical movement of the glove support 104 in the z-axis direction.

Detents 108 are spaced a distance D1 apart, where D1 is measured as the distance between the surfaces that are orthogonal to the plane of the housing saddle 103 and face each other. In embodiments, D1 is from about 0.25 inch to about 4.5 inches; about 0.25 inch; about 0.333 inch; about 0.5 inch; about 0.666 inch; about 0.75 inch; about 0.8 inch; about 1 inch; about 1.5 inches; about 2 inches; about 2.5 inches; about 3 inches; about 3.5 inches; about 4 inches; or about 4.5 inches. Other distances are possible.

Housing saddle 103 optionally includes receiving cavities 110 for securing pins. The example shown in FIG. 4 includes receiving cavities 110 for two securing pins, although other embodiments can be configured to receive more or fewer than two securing pins. The cavities can be threaded.

In other embodiments, housing saddle 103 includes two additional bottom detents at or near the position of the illustrated receiving cavities 110. The bottom detents, not shown in FIG. 4, are in place of, or in addition to, securing pins and receiving cavities 110. The bottom detents can have a similar shape to the top detents 108, but are spaced further apart and are larger than the top detents 108. The bottom detents can be adjacent to the bottom edge surface 114 or the side edge surfaces 116.

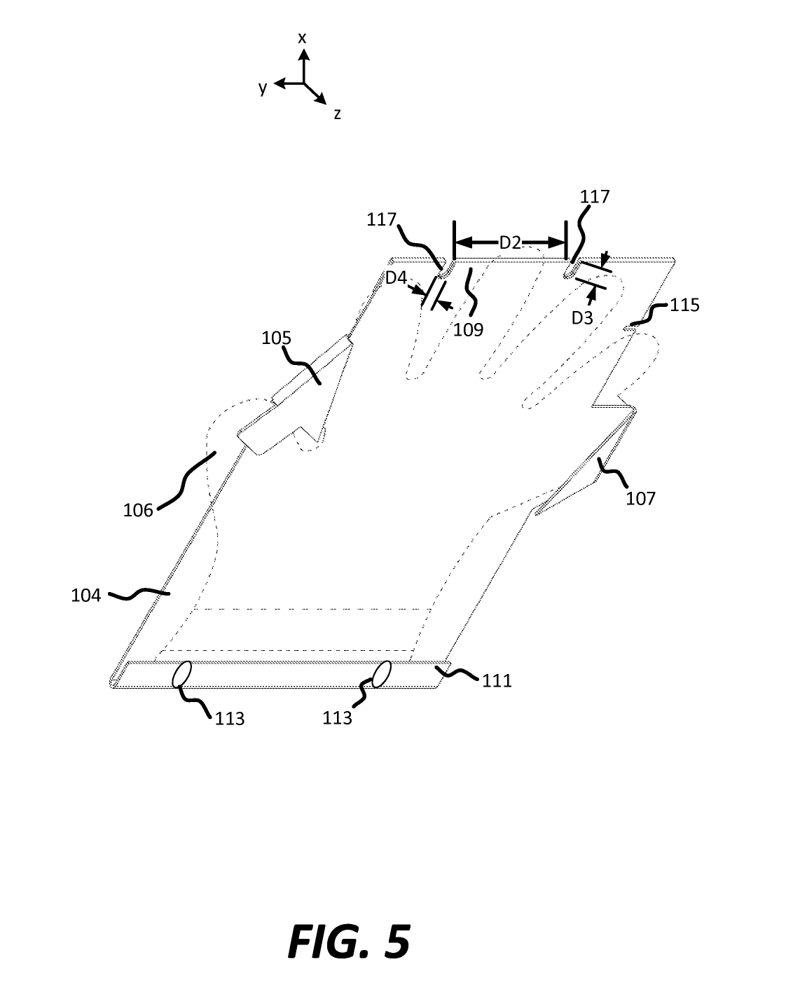

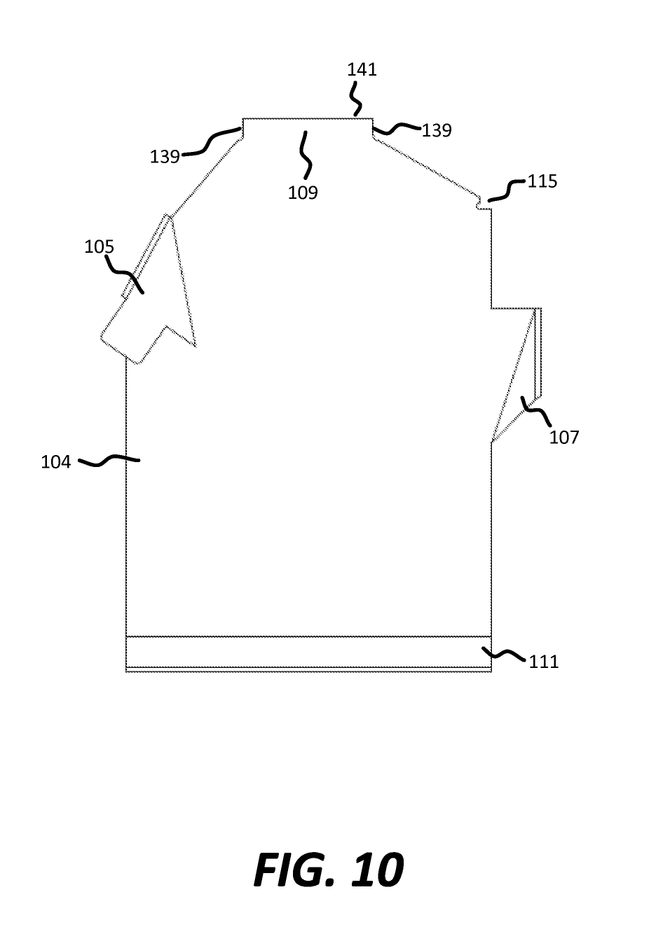

FIG. 5 illustrates the example glove support 104 and glove 106 shown in FIG. 4. The glove support 104 includes a first tab 105, a second tab 107, a third tab 111, insert tab 109, securing pin slots 113, band slot 115, and detent slots 117. Alternate embodiments of the glove support 104 and glove 106 are shown in FIGS. 10-14. Additional embodiments of glove supports are shown in U.S. patent application Ser. No. 29/523,839, the entirety of which is hereby incorporated by reference. Other embodiments can include more or fewer components.

The glove support 104 optionally includes one or more tabs. As illustrated, the glove support 104 includes a first tab 105. The first tab 105 is positioned over part of the first and second digits of the glove, and includes an optional extension that is positioned over at least part of the first digit of the glove.

The glove support 104 also includes a second tab 107 positioned near the fifth digit of the glove, and a third tab 111 positioned near the base of the glove. The tabs 105, 107 and 111 include a substantially planar surface in the x-y plane. Other embodiments include more or fewer tabs, or tabs in different locations.

The tabs 105, 107, and 111, provide resistance against the user's U pulling motion, which can facilitate removal of a glove from a stack of gloves in the glove support 104 along perforation lines. Tabs 105 and 107 can also help hold the glove in position while the unit is mounted on a wall, in a vertical position.

An insert tab 109 is integral with the glove support 104. Insert tab 109 has a width D2 that is less than or about equal to D1, the distance between the parallel, inner surfaces of detents 108. In this way, the outer surfaces of insert tab 109 can form an interference fit with the inner surfaces of detents 108.

In the glove support 104, detent slots 117 are adjacent to, and formed partially by, insert tab 109. The detent slots 117 have a depth of D3 and a width of D4. The depth D3 is about equal to or greater than the depth of detents 108. The width D4 is about equal to or greater than the width of detents 108. Detent slots 117 can provide additional lateral stability to the glove support 104. The detent slots 117 are optional.

The glove support 104 optionally includes securing pin slots 113. In embodiments where securing pins are used, securing pin slots 113 enable part of the securing pins to pass through the glove support 104 and engage with the glove dispensing housing 102.

Band slot 115 is an optional feature of glove support 104. In embodiments, an elastic band, such as a rubber band, is supported in part by band slot 115 and by the insert tab 109 or one of the detent slots 117. The elastic band stretches across, for example, the fourth digit of the glove or the third and the fourth digits of the gloves. The elastic band can inhibit glove sag, for example, when the glove dispensing housing 102 is positioned against a wall. In embodiments, the gloves 106 are secured to the glove support 104 near the base of the hand portion of the glove. Thus, when the glove dispensing housing 102 is not positioned parallel to the ground, there is a gravitational force pulling the fingertips of the gloves and the elastic band can counteract this gravitational force.

FIG. 6 illustrates an example detent 108. As illustrated, the detent 108 includes a top surface 125, a side surface 127, and a back surface 129. The detent has height H1. Also shown in FIG. 6 are top edge surface 112 and supporting surface 131 of the glove dispensing housing 102. In other embodiments, the detent 108 has more or fewer surfaces, and the surfaces can be curved or a different shape. Typically, the housing saddle 103 includes two detents 108, where a second detent, not shown in FIG. 6, is a mirror image of the detent 108 shown in FIG. 6.

The side surface 127 and back surface 129 can inhibit lateral movement of the glove support 104. Both are substantially planar and have a thickness. The detents 108 are made of a rigid material, such as a plastic or a metal. In embodiments, back surface 129 is integral to the top edge surface 112 of the housing saddle. Alternatively, back surface 129 is separate from the top edge surface 112, as shown in FIG. 6.

The top surface 125 of the detent inhibits vertical movement of the glove support 104. The height H1 of the top surface 125 of the detent corresponds to the thickness of the glove support 104, where H1 is about equal to or greater than the thickness of the glove support 104.

The top surface 125 of the detent is generally parallel to the supporting surface 131 of the glove dispensing housing 102. In other embodiments, the top surface 125 of the detent is angled oblique to the supporting surface 131 of the glove dispensing housing 102.

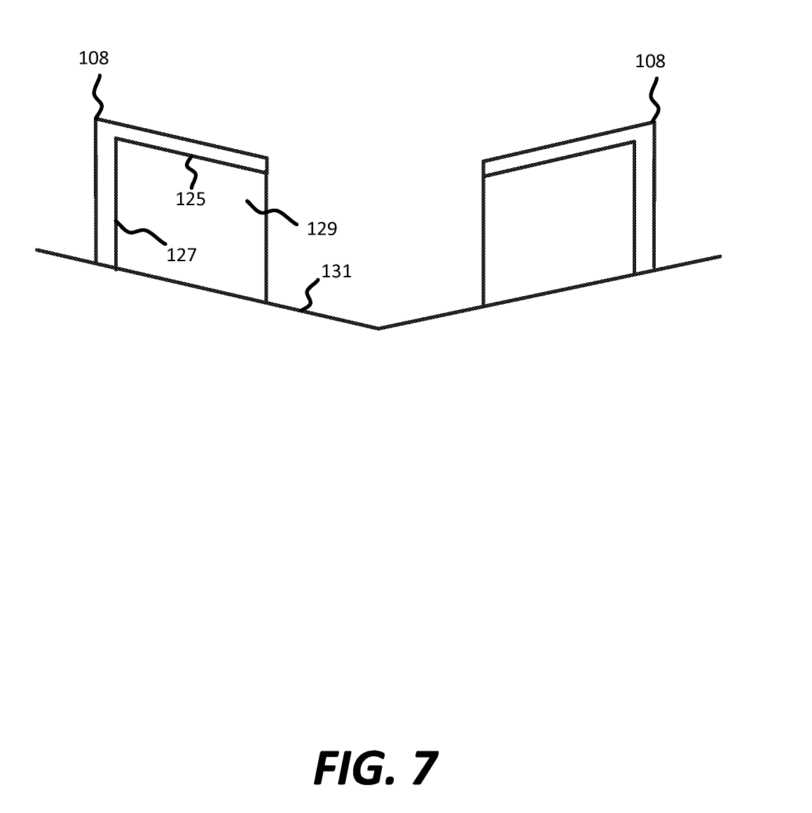

FIG. 7 shows a front view of two detents 108 positioned on a supporting surface 131 of the glove dispensing housing 102. In the embodiment shown, the supporting surface 131 has a v-shaped cross section near the detents 108. The top surface 125 of both detents is parallel to the supporting surface 131 below, but the top surfaces 125 are not co-planar. In other embodiments, the top surfaces 125 are co-planar and not parallel to the supporting surface 131.

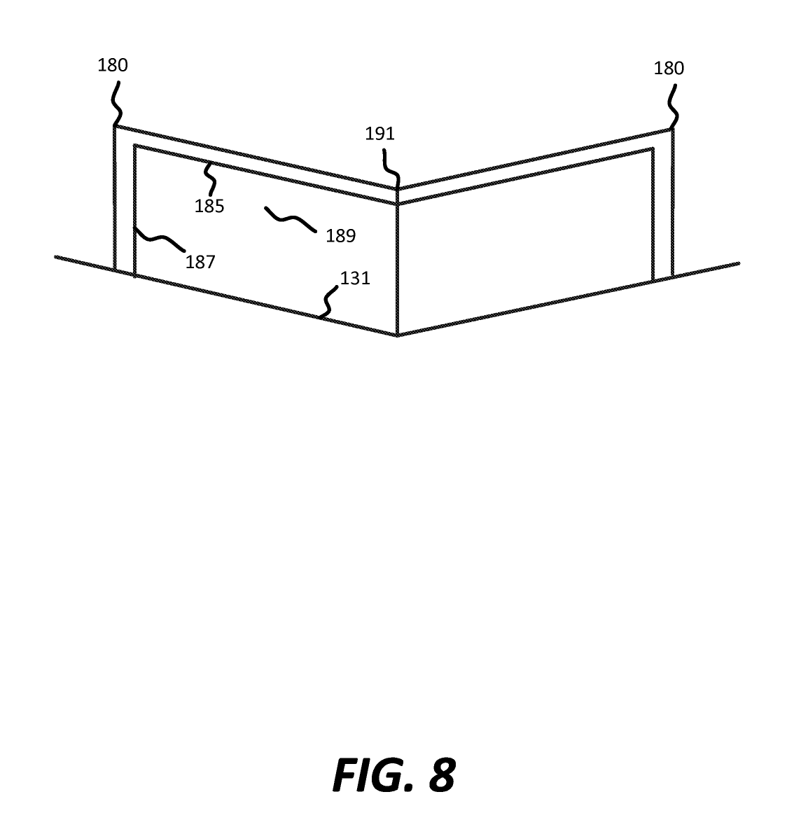

FIG. 8 shows a front view of an alternate embodiment of detent 180. The detents 180 include top surface 125, side surface 187, and back surface 189 of the detent, as well as seam 191. The detents are joined along seam 191, creating a continuous structure. In other embodiments, the top surface 125 does not have a v-shaped cross-section. The top surface can be “A”-shaped or a different curved surface in other embodiments.

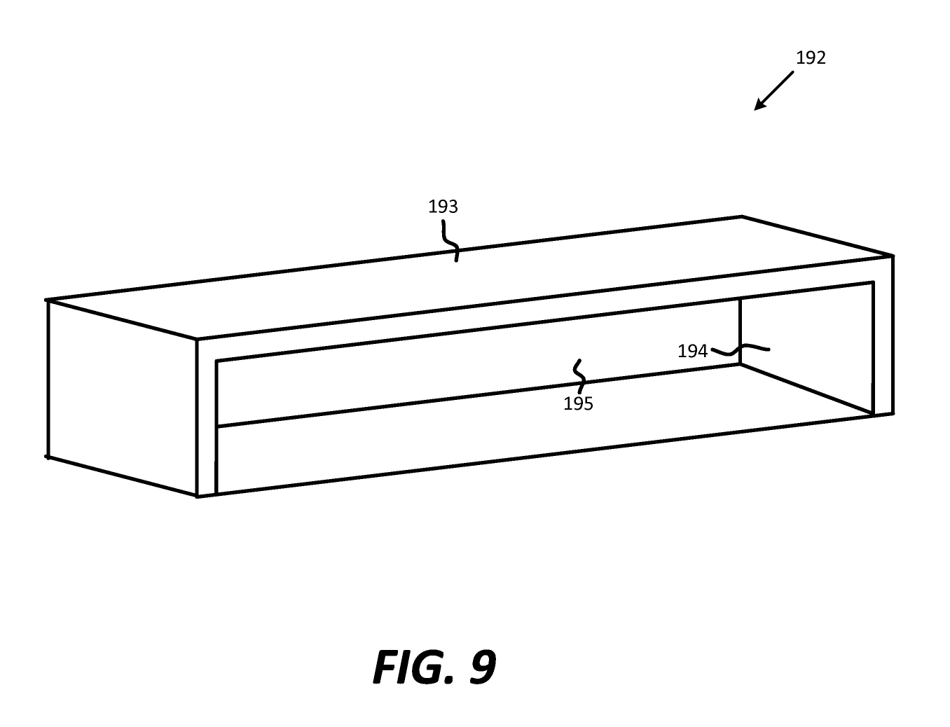

FIG. 9 shows another alternate embodiment of detent 192. The detent 192 includes a planar top surface 193, side surface 194, and back surface 195 of the detent. This detent 192 can be used in embodiments where the supporting surface of the glove dispensing housing 102 is flat and does not have any curvature.

FIG. 10 illustrates an alternate embodiment of the glove support 104. In contrast to the embodiment shown in FIGS. 4 and 5, the embodiment of glove support 104 in FIG. 10 does not include detent slots 117. Similar to the other embodiments, the outer, parallel surfaces 139 and front surface 141 of the insert tab 109 contact the inner surfaces of detents 108. Also of note is that this embodiment does not include securing pin slots 113.

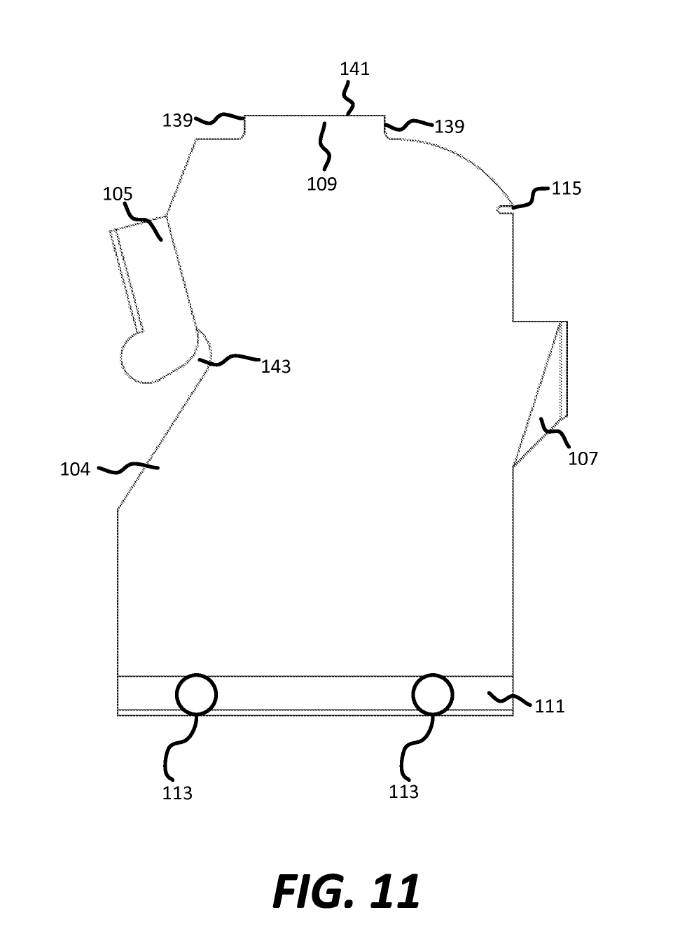

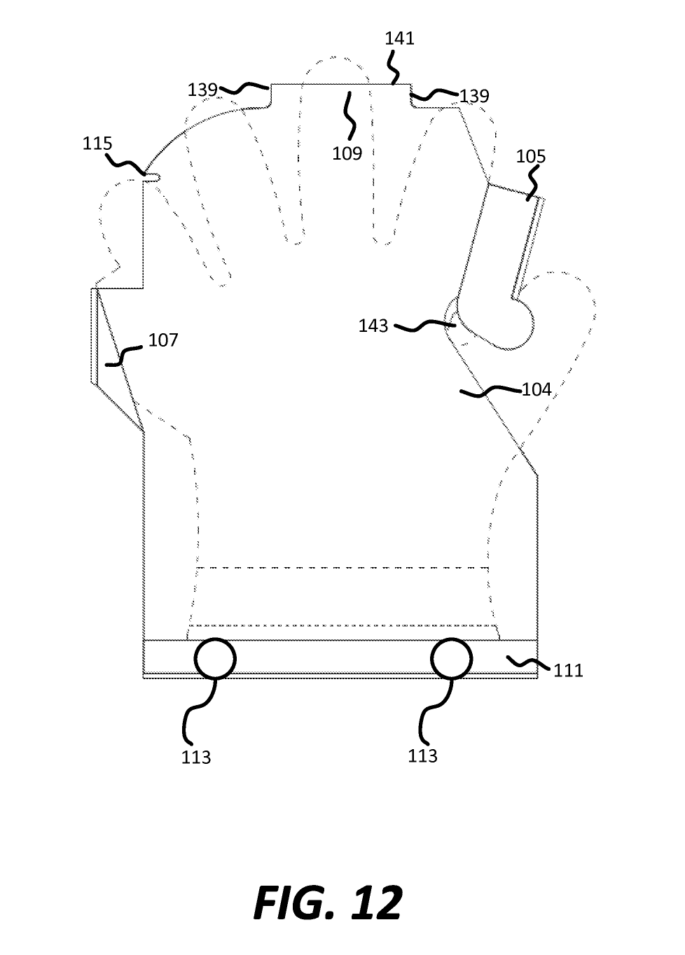

FIG. 11 illustrates another alternate embodiment of the glove support 104. Similar to the embodiment shown in FIG. 10, this embodiment does not include detent slots 117 but does include the securing pin slots 113. An embodiment configured to support left-handed gloves is shown in FIG. 12. Other configurations are possible.

In this embodiment, the shape of the first tab 105 differs from the embodiments in FIGS. 4 and 10. The first digit tab 105 is more rounded than in those embodiments. The rounded shape can lessen the possibility of puncturing the glove when a user is removing a glove from the glove assembly, as compared to the more pointed shape shown in FIGS. 4 and 10.

Another difference is the curved void 143 beneath the first digit tab 105. The curved shape of the first digit tab 105 can be die-cut. Then, when that portion is folded over, the first digit tab 105 moves from the plane of the glove support 104 to parallel to the plane of the glove support 104 in the position shown in FIG. 11. This configuration reduces the size of the card stock that is being die-cut.

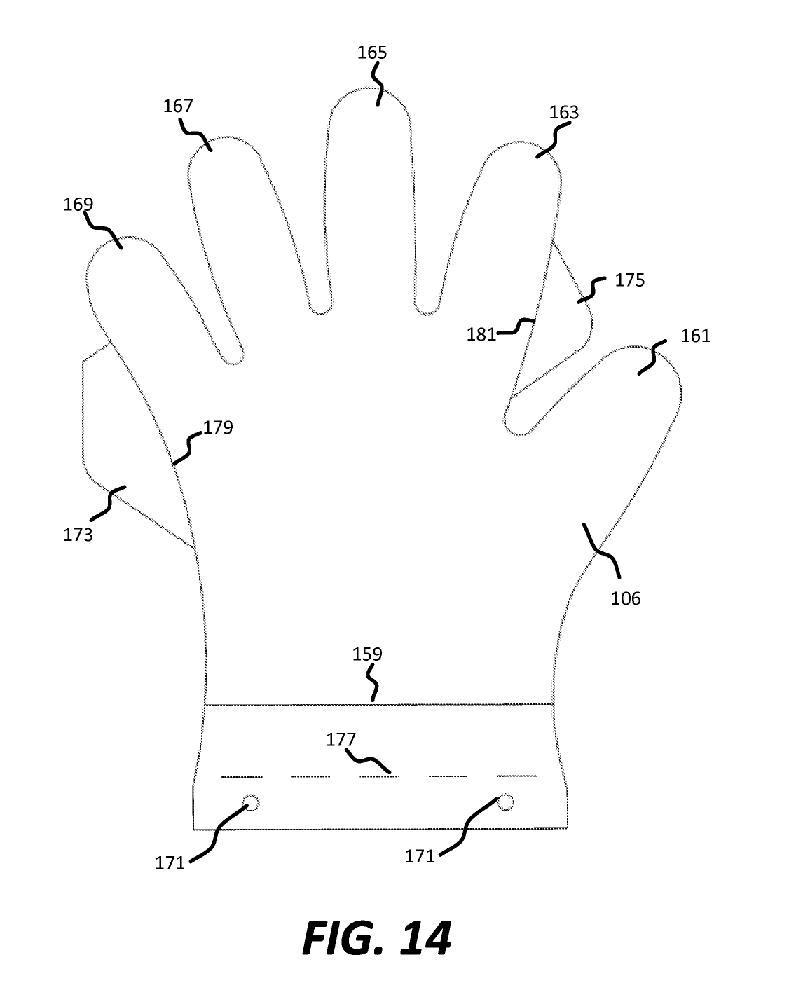

FIG. 13 illustrates an example glove 106. The glove 106 includes an opening 159, a first digit 161, a second digit 163, a third digit 165, a fourth digit 167, a fifth digit 169, slots for a securing pin 171, a fifth digit tab 173, a second digit tab 175, a base perforation line 177, a fifth digit perforation line 179, and a second digit perforation line 181. Example of other embodiments are shown in U.S. patent application Ser. No. 29/523,847, the entirety of which is hereby incorporated by reference. As shown, the glove 106 is configured for a right hand to be inserted. In other embodiments the glove 106 is configured for a left hand to be inserted, as shown in FIG. 14. Other embodiments can include more or fewer components.

A user U inserts their hand into the glove 106 through opening 159. Then the user U positions their fingers in the corresponding digits of the glove 106. As illustrated, the glove 106 includes five digits: a first digit 161, a second digit 163, a third digit 165, a fourth digit 167, and a fifth digit 169. Other embodiments include just three digits (a “lobster” style glove) or just one digit (a “bag” style glove).

After the user's U hand is in the glove, the user U applies a force sufficient to cause the glove to separate from the stack of gloves along the perforation lines 177, 179 and 181. In embodiments, the torn-away glove does not include tabs 173 and 175. In other words, portions of the glove 106 remain within the glove support 104 after the user U tears away the top-most glove in the stack, such as tabs 173 and 175, as well as a portion near the bottom perforation line 177. The user can perform the aforementioned steps in a single, quick motion.

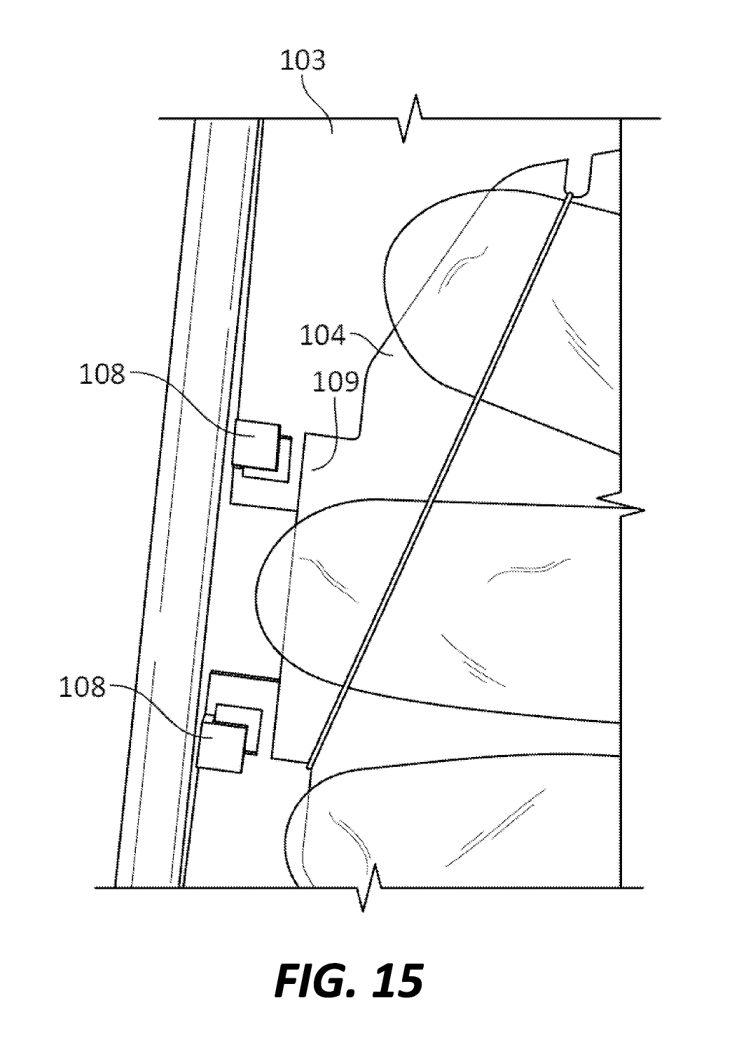

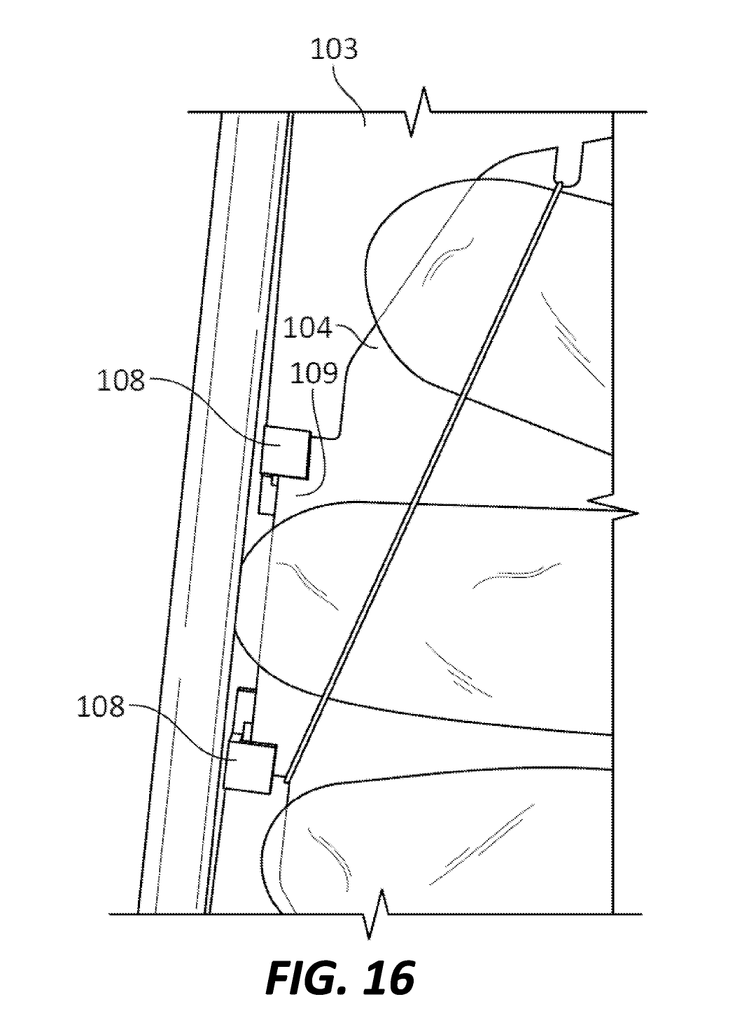

FIGS. 15 and 16 illustrate un-mated and mated conditions, respectively, between the glove support 104 and housing saddle 103. In the embodiment shown, the bottom tab 111 has first been secured to the housing saddle 103, by, for example, securing pins or by mating with detents on the housing saddle 103. In other embodiments, the mating process in FIGS. 15 and 16 occurs first and then the bottom tab 111 is secured to the housing saddle 103. Also, the depicted embodiment includes an optional elastic band that stretches across two digits of the glove. Other embodiments do not include the elastic band or have the elastic band configured differently.

FIG. 15 shows the insert tab 109 of glove support 104 as it approaches detents 108 of the housing saddle 103. The insert tab 109 has not formed an interference fit with the detents 108 in the position shown in FIG. 15. Also, the illustrated embodiment of glove support 104 does not include detent slots 117. FIG. 16 shows the insert tab 109 mated with the detents 108. When mated, lateral and vertical movement of the glove support 104 is nearly or completely arrested.

The block diagrams depicted herein are just examples. There may be many variations to these diagrams described therein without departing from the spirit of the disclosure. For instance, components may be added, deleted or modified.

The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application. Regardless whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the claimed invention and the general inventive concept embodied in this application that do not depart from the broader scope.

What is claimed is:

1. A glove support, comprising:
a body with a planar surface including a mating tab,
wherein the mating tab is configured to form an interference fit with a detent on a housing; and
wherein the body is configured to support a plurality of gloves; and
at least one holding tab, the at least one holding tab being integral with the body and having a planar surface that is substantially parallel to the planar surface of the body.

2. The glove support of claim 1, wherein each of the plurality of gloves include five digits; and
wherein the at least one holding tab includes a first holding tab that is positioned over at least a portion of a first digit of the gloves.

3. The glove support of claim 2, wherein the at least one holding tab includes a second holding tab that is positioned over at least a portion of a fifth digit of the gloves.

4. The glove support of claim 2, wherein the at least one holding tab includes a second holding tab that is positioned over at least a portion of a base of the plurality of gloves.

5. The glove support of claim 4, wherein the at least one holding tab includes a third holding tab that is positioned over at least a portion of a fifth digit of the gloves.

6. The glove support of claim 5, wherein the glove support further comprises at least one securing pin slot located near a bottom edge of the glove support.

7. The glove support of claim 2, wherein the glove support further comprises a band slot.

8. The glove support of claim 2, wherein the glove support further comprises at least one mating slot, the at least one mating slot positioned adjacent to the mating tab.

9. The glove support of claim 8, wherein the at least one mating slot is configured to receive a portion of the detent and form the interference fit with the detent.

10. The glove support of claim 8, wherein the first holding tab includes an extension over at least part of the first digit of the glove.

11. A glove dispensing system, comprising:
a body with a planar surface including a mating tab, the mating tab including at least two outer edge surfaces, wherein the at least two outer edge surfaces are substantially normal to the planar surface of the body, and wherein the at least two outer edge surfaces are substantially parallel to each other;
at least one holding tab, the at least one holding tab being integral with the body and having a planar surface that is substantially parallel to the planar surface of the body;
wherein the body is configured to support a plurality of gloves; and
wherein at least one glove is supported by the body.

12. The glove dispensing system of claim 11, wherein the at least one glove includes five digits; and
wherein the at least one holding tab includes a first holding tab that is positioned over at least a portion of a first digit of the gloves.

13. The glove dispensing system of claim 12, wherein the at least one holding tab includes a second holding tab that is positioned over at least a portion of a fifth digit of the gloves; and
wherein the at least one holding tab includes a third holding tab that is positioned over at least a portion of a base of the plurality of gloves.

14. The glove dispensing system of claim 13, wherein the body further comprises a band slot; and
wherein the at least one glove includes a first perforation line adjacent to a second digit and a second perforation line adjacent to the fifth digit.

15. The glove dispensing system of claim 13, wherein the body further comprises at least one mating slot, the at least one mating slot positioned adjacent to the mating tab; and
wherein the at least one mating slot is configured to receive a detent and form an interference fit with the detent.

16. A glove tray, comprising:
a body including an insert tab, a first resistance tab, a second resistance tab, and a third resistance tab,
wherein the insert tab is integral to the body and configured to form an interference fit with a securing component;
wherein the body is substantially planar; and
wherein each of the first resistance tab, the second resistance tab, and the third resistance tab are substantially planar, supported by the body, and parallel to the planar surface of the body.

17. The glove tray of claim 16, wherein the glove tray is configured to receive a plurality of gloves;
wherein each of the plurality of gloves includes five digits;
wherein the first resistance tab is positioned over at least a portion of a first digit of the gloves;
wherein the second resistance tab is positioned over at least a portion of a fifth digit of the gloves; and
wherein the third resistance tab is positioned over at least a portion of a base of the plurality of gloves.

18. The glove tray of claim 17, wherein the body further includes a band slot; and
wherein the body and the third resistance tab both include a securing pin slot.

19. The glove tray of claim 17, wherein the glove tray further comprises at least one mating slot, the at least one mating slot positioned adjacent to the insert tab; and
wherein the at least one mating slot is configured to receive a detent and form the interference fit with the detent.

20. The glove tray of claim 19, wherein the first resistance tab includes an extension over at least part of the first digit of the glove;
wherein the body further includes a band slot; and
wherein the body and the third resistance tab both include at least one securing pin slot, wherein the at least one securing pin slot in the body shares a central axis with the at least one securing pin slot in the third resistance tab.
